(19) United States
(12) Patent Application Publication
(10) Pub. No.: US 2025/0384867 A1
(43) Pub. Date: Dec. 18, 2025

(54) SYSTEM AND METHOD FOR EMULATING HUMAN CONVERSATION WITH PETS

(71) Applicant: Dustin Nephi Slade, Cody, WY (US)

(72) Inventor: Dustin Nephi Slade, Cody, WY (US)

(21) Appl. No.: 19/240,796

(22) Filed: Jun. 17, 2025

Related U.S. Application Data
(60) Provisional application No. 63/660,833, filed on Jun. 17, 2024.

(51) Int. Cl. G10L 13/00; A61B 5/01; A61B 5/0205; A61B 5/024; A61B 5/08; A61B 5/16; G10L 15/18

(52) U.S. Cl. CPC G10L 13/00; A61B 5/01; A61B 5/02055; A61B 5/024; A61B 5/0816; A61B 5/165; G10L 15/1822

(57) ABSTRACT

The present invention relates to the field of human-animal interaction, with certain embodiments utilizing artificial intelligence (AI) and Internet of Things (IoT) technologies to emulate human conversation with pets. In particular, preferred embodiments of the present invention are related to a system and method for creating interactive, real-time conversations between pets and their owners via a computerized system.

### CROSS REFERENCE TO RELATED APPLICATIONS

This application claims priority to U.S. Provisional Patent Application No. 63/660,833, filed Jun. 17, 2024, the entirety of which is incorporated by reference herein.

### TECHNICAL FIELD

The present invention relates to the field of human-animal interaction, with certain embodiments utilizing artificial intelligence (AI) and Internet of Things (IoT) technologies to emulate human conversation with pets. In particular, preferred embodiments of the present invention are related to a system and method for creating interactive, real-time conversations between pets and their owners via a computerized system.

### BACKGROUND

Pet owners often seek ways to bond with their pets, entertain them, and alleviate loneliness. Traditional methods of interaction, while fulfilling, do not offer the dynamic and responsive engagement that technology can provide. Existing solutions in the market are limited to basic communication tools that do not simulate conversational interactions.

While pet owners frequently speak with their pets, and certain pets can understand basic commands or tasks given with verbal cues, nearly all pets are incapable of providing a verbal response. Even still, pets are capable of mimicking or displaying emotions and actions in response to verbal communications from their owners.

Even though most pet owners are content with the affection their pets provide, there are many pet owners that would still prefer to be able to better communicate with their pets. Still other pet owners would actually like to be able to have back and forth communications with their pet in a spoken language.

Therefore, there is a need in the art for a system and method that allows pet owners to engage in realistic and interactive conversations with their pets. This can be achieved by leveraging pre-trained natural language models, speech-to-text, and text-to-speech technologies integrated into a portable, Bluetooth-enabled speaker device that attaches to a pet's collar or that itself comprises a pet's collar.

### SUMMARY OF THE INVENTION

Preferred embodiments of the present invention are directed to automated systems and methods for integrating conversational functionality using pre-trained natural language models, speech recognition, and synthesis technologies. This combination allows for the creation of a real-time interactive communication system between pets and their owners.

According to an embodiment of the present invention, a system for creating interactive, real-time conversations between pets and their owners via a computerized system comprises: a pet communication device, comprising a processor, a data communications module and a pet communication module stored in non-transitory memory and configured to instruct said processor to: detect engagement of a directed conversation; process initial speech associated with the directed conversation; determine a sentiment analysis of the initial speech; generate a response to the initial speech, based at least in part on the sentiment analysis and known data points associated with a pet associated with the pet communication device; and deploy an audio response, based on the response to the initial speech, to an audio playback component.

According to an embodiment of the present invention, the system may be further configured to receive sensor data associated with the pet from the sensor module; process an animal state, based at least in part on the sensor data; and incorporate said animal state into the known data points associated with the pet.

According to an embodiment of the present invention, the sensor module is selected from the group comprising, an optical sensor module, an audio sensor module, an infrared sensor module, accelerometer module, a heart rate sensor module, a respiratory rate sensor module, an electrochemical sensor module, or a thermal sensor module.

According to an embodiment of the present invention, data related to the animal state is based at least in part on a delta between a first animal state occurring prior to receipt of the initial speech and a second animal state occurring after the receipt of the initial speech.

According to an embodiment of the present invention, the system is further configured to: detect changes in affect of a user, based at least in part on said audio response; and analyze the changes in affect for desirability of outcome; and update a data model for use in generating response to user to pet speech based at least in part on the desirability of outcome.

According to an embodiment of the present invention, the system is further configured to: transmit, via the data communications module, the initial speech to a remote computing device for processing; and receive, via the data communications module, the response to the initial speech, which was processed on the remote computing device.

According to an embodiment of the present invention, the audio playback component is located on a user device. In an alternate embodiment, the audio playback component is located on the pet communication device. The audio playback component may reside on the user device for a wide variety of reasons, including but not limited to privacy or when the pet is in a noisy environment. The audio playback component may reside on the pet communication device for a wide variety of reasons, including but not limited to, augmenting the illusion of pet-owner communication.

According to an embodiment of the present invention, a computerized method creating interactive, real-time conversations between pets and their owners comprises the steps of: detecting, via a pet communication device, engagement of a directed conversation; processing, via a pet communication device, initial speech associated with the directed conversation; determining, via a pet communication device, a sentiment analysis of the initial speech; generating, via a pet communication device, a response to the initial speech, based at least in part on the sentiment analysis and known data points associated with a pet associated with the pet communication device; and deploying an audio response, based on the response to the initial speech, to an audio playback component.

According to an embodiment of the present invention, the method further comprises the steps of: receiving sensor data associated with the pet from the sensor module; processing an animal state, based at least in part on the sensor data; and incorporating said animal state into the known data points associated with the pet.

According to an embodiment of the present invention, the method further comprises the steps of: detecting changes in affect of a user, based at least in part on said audio response; analyzing the changes in affect for desirability of outcome; and updating a data model for use in generating response to user to pet speech based at least in part on the desirability of outcome.

According to an embodiment of the present invention, the method further comprises the steps of: transmitting, via a data communications module, the initial speech to a remote computing device for processing; and receiving, via the data communications module, the response to the initial speech, which was processed on the remote computing device.

Embodiments of the present invention may include an application that communicates with a speaker device which is configured to communicate wirelessly with an application on a mobile device or other computing device, and the speaker device may be attached to a pet's collar, harness, or other article attached to the pet. Preferred embodiments of the system interpret an owner's speech, formulates appropriate responses, converts these responses to speech, and delivers them through the speaker device, creating the illusion of a conversation with the pet.

### BRIEF DESCRIPTION OF THE DRAWINGS

Accompanying this written specification is a collection of drawings of exemplary embodiments of the present disclosure. One of ordinary skill in the art would appreciate that these are merely exemplary embodiments, and additional and alternative embodiments may exist and still within the spirit of the disclosure as described herein.

FIG. 1 is a schematic illustration of an exemplary computing device, in accordance with at least some exemplary embodiments of the present invention;

FIG. 2 is a schematic illustration of an exemplary network, in accordance with at least some exemplary embodiments of the present invention;

FIG. 3 is a depiction of an exemplary embodiment of a speaker device that may be utilized with embodiments of the present invention;

FIG. 4 is an illustration of an exemplary method for emulating human conversations with animals, in accordance with an embodiment of the present invention;

FIG. 5 is an illustration of an exemplary method for emulating human conversations with animals, in accordance with an embodiment of the present invention; and

FIG. 6 is an illustration of an exemplary device for emulating human conversations with animals as being worn by a canine, in accordance with an embodiment of the present invention.

### DETAILED DESCRIPTION

According to one embodiment of the present invention, a system for emulating human conversation with pets may be comprised of a set of computer-enabled modules that provide for the ability to interpret, generate, and synthesize speech based on pre-trained natural language models.

In certain embodiments, the system involves a mobile application that interfaces with a network connected (e.g., Bluetooth, WIFI, NFC, cellular) speaker device. The device may be configured to be attached to a pet, such as via a collar, harness, clothing, or other article capable of being attached to the pet.

According to certain embodiments of the present invention, an exemplary method involved in the conversation flow enabled by the system may include: capturing speech input from an owner via a input device, such as being captured by an application on a mobile device, or a microphone on the network connected speaker device; processing of the speech input, such as via the mobile device, or via the speaker device; converting the input to a human recognizable format, such as audio or text; interpreting, such as via a pre-trained natural language model the human recognizable format; and generating a human recognizable response, such as audio or text.

In preferred embodiments, the resulting human recognizable response (e.g., synthesized speech) is then transmitted to the speaker device via an appropriate transmission means (e.g., Bluetooth, WIFI, cellular), creating the interactive conversational experience.

Preferred embodiments of the speaker device are lightweight (e.g., weighing less than eight ounces) and compact (e.g., no larger than three cubic inches), with a speaker quality sufficient to emulate authentic real-time conversation. Preferred embodiments of the present invention may also include a loop for attachment to a pet's collar, harness, clothing or other article attachable to a pet. One of ordinary skill in the art would appreciate that there are numerous shapes, sizes and means for attaching embodiments of the present invention to a pet, and given the various sizes of pets, sizes and capabilities of the speaker device may vary in appropriate manners. For instance, a larger device may be used for a larger pet, such as a great dane, and a smaller device might be used for a smaller pet, such as a rabbit or chihuahua. For clarity, the term pet is intended to include exotic pets, such as elephants and mice, and devices may be appropriately sized for any size pet.

In certain embodiments, a software application portion utilized with the systems described herein may feature various personality profiles, allowing pet owners to choose or customize conversational styles of responses. In preferred embodiments, these profiles can be created using pre-trained natural language models fine-tuned with datasets from literature, cinema, and other sources. In still further embodiments, users and pet owners may influence the type and nature of these profiles, by prompting the models with desired inputs and ultimately providing for profiles based on multiple sources, creating entirely new profiles based on combinations of various datasets and sources.

According to preferred embodiments of the present invention, the system may also be configured to process various conversational contexts, including commands and observations. Embodiments may be configured to provide generic, context-appropriate responses when specific commands or observations are not readily available or able to be interpreted accurately.

In certain preferred embodiments, the system may include bioinformatic components and/or modules to assess a pet's physiological state and integrate this data into the conversation flow, enhancing the realism and responsiveness of the interactions. For instance, sensors, such as heart rate monitors, heart rate variability sensors, oxygen sensors, respiratory rate sensors, vibration sensors, electrochemical sensors, or any combination thereof, could be used to determine physiological or psychological states of the pet in order to optimize responses. One of ordinary skill in the art would appreciate that there are numerous sensor, module and bioinformatic components that could be used with embodiments of the present invention, and embodiments of the present invention are contemplated for use with any such components, sensors and modules.

In certain embodiments, the system may also be configured to store conversation history, allowing the natural language model to recall previous interactions, thus providing a more coherent and contextually aware conversational experience. This may also include embodiments where the system receives input from a user regarding their experience with the existing profile related to the pet, and update that profile according to the user's perceived or provided experience with the profile. In some embodiments, this may include receiving input from a user's devices or wearables that track certain bioinformatics or other information about the user, such as heart rate, respiratory rate, pupil dilatation, voice tone, or other data point or combination of data points. One of ordinary skill in the art would appreciate that there are numerous data points that could be used, and embodiments of the present invention are contemplated for use with any such data points.

In accordance with one embodiment of the present invention, the system may be configured with modules able to provide analysis of the digitally collected data from various sources using artificial intelligence or machine learning means, including, but not limited to, machine learning models trained on various amounts of test and training data, neural networks (e.g., Artificial Neural Networks (ANN), Convolution Neural Networks (CNN), Recurrent Neural Networks (RNN)), deep learning models and deep-learning-based generative models (e.g., generative adversarial networks (GANs)). One of ordinary skill in the art would appreciate that there are numerous types of ML and AI systems that could be used for the purposes detailed herein, and embodiments of the present invention are contemplated for use with any such ML or AI system. This also helps assist with the predictive analysis and creation of the responses for the speaker device to generate. The system may also be configured to provide analysis that delivers notifications regarding predicted responses, and receive updates and input on the quality and appropriateness of the generated responses.

According to an embodiment of the present invention, the system may be configured to use location and positioning data to adjust pet responses based on proximity and orientation relative to owner. Location and positioning data, may be obtained, for instance, via GPS or Bluetooth proximity sensors, for example, adjust responses based on the pet's geographic location or location relative to the owner. For instance, if the pet is detected in an unfamiliar location (e.g., a park), the system may generate an encouraging response, whereas spatial proximity to the owner may trigger a more familiar tone. One of ordinary skill in the art would appreciate that there are numerous ways to determine location and position, and embodiments of the present invention are contemplated for use with any appropriate means for determining location and position, including, but not limited to, spatial data. Location and positioning data may be utilized for both the speaker device, and any software application or audio device operated by the user (e.g., headphones).

According to an embodiment of the present invention, the system may be configured to use various AI/ML systems (e.g., stable diffusion, DALL-E) to generate images to present images to pet (e.g., via google glass for pet or a video screen installed (e.g., above food bowl) so that owner feels that there is an additional realistic component to the interaction. In these embodiments, the pet also gets some higher level of meaning as well from the system-augmented conversation.

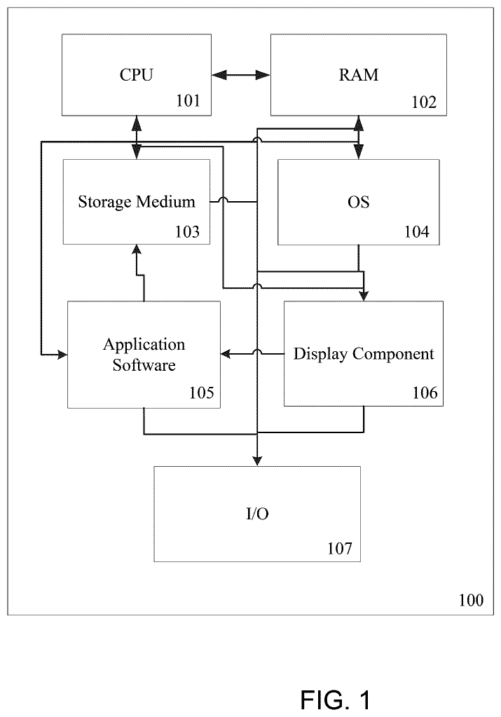

Turning now to FIG. 1, an illustrative representation of a computing device appropriate for use with embodiments of the system of the present disclosure is shown. The computing device 100 can generally be comprised of a Central Processing Unit (CPU, 101), optional further processing units including a graphics processing unit (GPU), a Random Access Memory (RAM, 102), a mother board 103, or alternatively/additionally a storage medium (e.g., hard disk drive, solid state drive, flash memory, cloud storage), an operating system (OS, 104), one or more application software 105, a display element (e.g., monitor, capacitive touchscreen) 106, and one or more input/output devices/means 107, including one or more communication interfaces (e.g., RS232, Ethernet, Wifi, Bluetooth, USB). Useful examples include, but are not limited to, personal computers, servers, tablet PCs, smartphones, or other computing devices. In preferred embodiments of the present invention, multiple computing devices can be operably linked to form a computer network in a manner as to distribute and share one or more resources, such as clustered computing devices and server banks/farms.

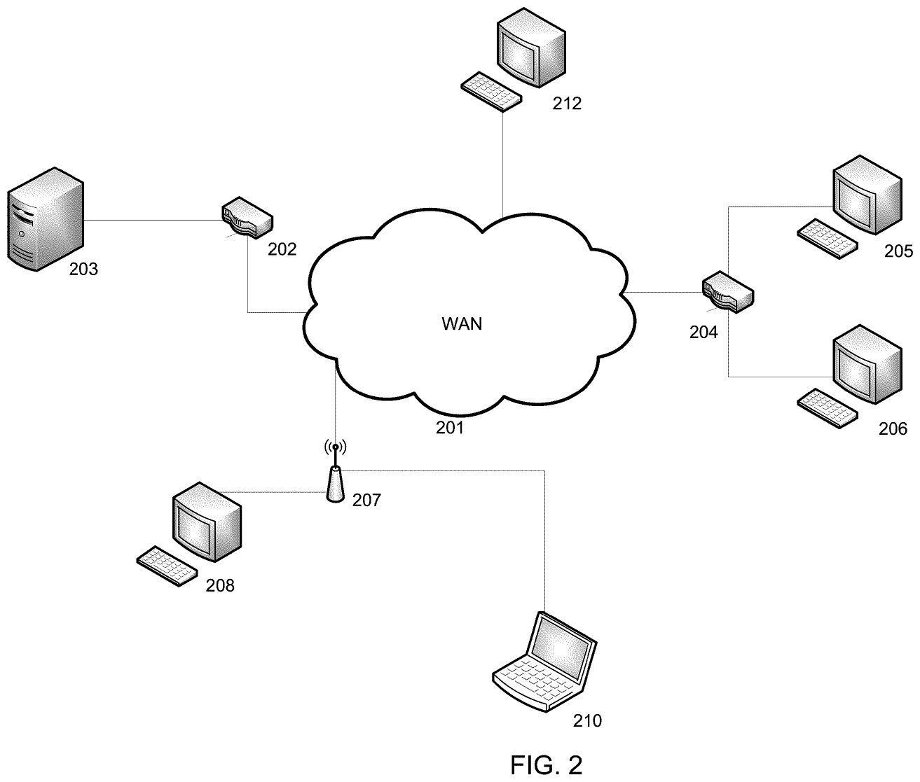

Various examples of such single-unit and multi-unit computer networks suitable for embodiments of the disclosure, their typical configuration and many standardized communication links are well known to one skilled in the art, as explained in more detail and illustrated by FIG. 2, which is discussed herein-below.

According to an exemplary embodiment of the present disclosure, data may be transferred to the system, stored by the system and/or transferred by the system to users of the system across local area networks (LANs) or wide area networks (WANs). In accordance with the previous embodiment, the system may be comprised of numerous servers, mining hardware, computing devices, or any combination thereof, communicatively connected across one or more LANs and/or WANs. One of ordinary skill in the art would appreciate that there are numerous manners in which the system could be configured and embodiments of the present disclosure are contemplated for use with any configuration.

Referring to FIG. 2, a schematic overview of a system in accordance with an embodiment of the present disclosure is shown. The system is comprised of one or more application servers 203 for electronically storing information used by the system. Applications in the server 203 may retrieve and manipulate information in storage devices and exchange information through a WAN 201 (e.g., the Internet). Applications in server 203 may also be used to manipulate information stored remotely and process and analyze data stored remotely across a WAN 201 (e.g., the Internet).

According to an exemplary embodiment, as shown in FIG. 2, exchange of information through the WAN 201 or other network may occur through one or more high speed connections. In some cases, high speed connections may be over-the-air (OTA), passed through networked systems, directly connected to one or more WANs 201 or directed through one or more routers 202. Router(s) 202 are completely optional and other embodiments in accordance with the present disclosure may or may not utilize one or more routers 202. One of ordinary skill in the art would appreciate that there are numerous ways server 203 may connect to WAN 201 for the exchange of information, and embodiments of the present disclosure are contemplated for use with any method for connecting to networks for the purpose of exchanging information. Further, while this application refers to high speed connections, embodiments of the present disclosure may be utilized with connections of any speed.

Components or modules of the system may connect to server 203 via WAN 201 or other network in numerous ways. For instance, a component or module may connect to the system i) through a computing device 212 directly connected to the WAN 201, ii) through a computing device 205, 206 connected to the WAN 201 through a routing device 204, or iii) through a computing device 208, 210 connected to a wireless access point 207. One of ordinary skill in the art will appreciate that there are numerous ways that a component or module may connect to server 203 via WAN 201 or other network, and embodiments of the present disclosure are contemplated for use with any method for connecting to server 203 via WAN 201 or other network. Furthermore, server 203 could be comprised of a personal computing device, such as a smartphone, acting as a host for other computing devices to connect to.

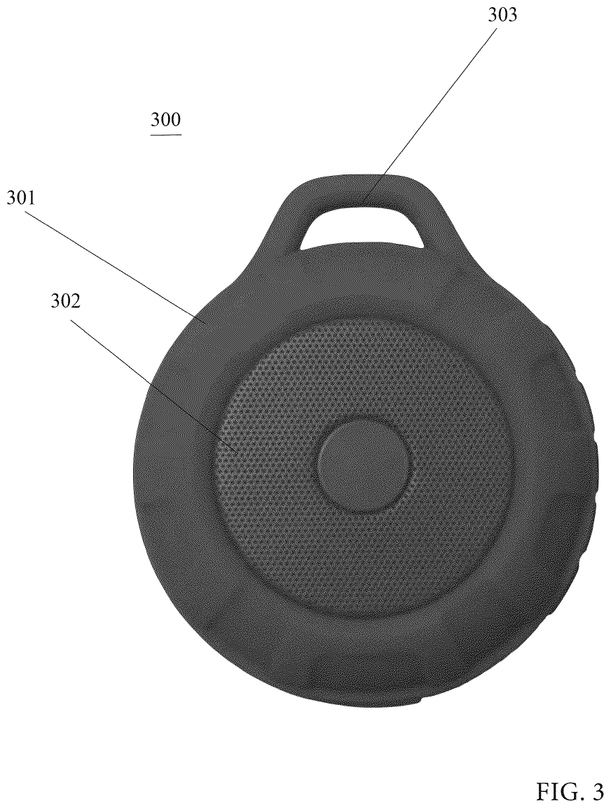

Turning to FIG. 3, an exemplary embodiment of the speaker device 300 used with the system is depicted. The speaker device 300 depiction is intended to be exemplary, and not limiting, and embodiments of the present invention are intended to be used with speaker devices of any shape, size or combination of components, depending on use case and other considerations. As depicted, speaker device 300 comprises a housing 301, a speaker unit 302 and an attachment feature 303. In preferred embodiments, the housing 301 is a protective shell that encloses the speaker device 300 and provides protection to the internal components of the device. The housing 301 may be comprised of a material that is robust enough to withstand the generally daily stresses of use with a pet. Appropriate material features for the housing include, but are not limited to, materials that are water resistant or waterproof, impact resistant or otherwise hardened, hypoallergenic to the pet, or any combination thereof. In further embodiments, the housing may be layered, with two or more layers, comprising: i) a softer or absorbent layer for reducing impact and physical stress against the electronic interior components of the speaker device 300; and ii) a more rigid layer for maintaining structure and protection for the more sensitive electronic interior components. Additional layers may be used, such as an exterior softer or hypoallergenic layer that is comfortable for daily use on the pet. One of ordinary skill in the art would appreciate that there are numerous types of materials and layers that could be utilized with embodiments of the present invention, and embodiments of the present invention are contemplated for use with any appropriate materials and layers.

The speaker device 300 may further comprise a speaker unit 302, which may comprise one or more sensors (e.g., audio, impact, accelerometer, optical, visual, thermal, electrochemical, infrared) input devices (e.g., microphone, touch pad), output devices (e.g., speaker, haptic feedback, illumination), or any combination thereof. One of ordinary skill in the art would appreciate that there are numerous types of sensors, input devices and output devices that could be used with embodiments of the present invention, and embodiments of the present invention are contemplated for use with any appropriate sensors, input devices, and output devices.

The speaker device 300 may further comprise an attachment feature 303. The attachment feature 303 is configured to allow the speaker device 300 to be secured to a pet, typically via some form of detachable attachment, such as a leash, collar, vest or other apparatus configured to secure to a pet. While as depicted the attachment feature 303 is located on top of the speaker device 300, in other embodiments, the attachment feature could be located anywhere on the speaker device 300, including the sides or back, and there could further be multiple attachment features 303 on a device, such as loops on the sides or back of the speaker device to thread ends of a collar on, in order to secure the speaker device to the collar. One of ordinary skill in the art would appreciate that there are numerous types of attachment features that could be utilized with embodiments of the present invention, and embodiments of the present invention are contemplated for use with any appropriate attachment feature.

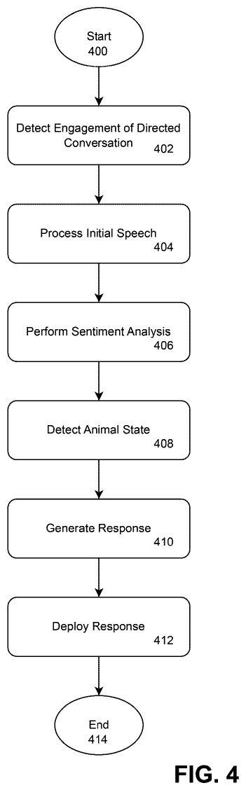

Turning now to FIG. 4, an exemplary method for emulating human conversations with animals, in accordance with an embodiment of the present invention, is shown. The process starts at Step 400, with the system being engaged to process conversations. At step 402, the system engages the directed conversation process. In certain embodiments, the system may be engaged, for instance, by a user engaging a computing device (e.g., via an application on a mobile device) in order to converse with their pet. In other embodiments, detection of a trigger, such as a word or phrase that would engage the system (e.g., pet's name). In still further embodiments, the system may be configured to automatically begin the process upon the system, or a component thereof, detecting an event that is a triggering event. For instance, a pet collar with optic or other vision system sensors may be configured to use one or more visual cues (e.g., facial recognition, eye tracking) to identify and confirm a user is attempting to engage in conversation with the pet. As another example, engagement may be detected by a user-activated toggle. One of ordinary skill in the art would appreciate there are numerous sensors and trigger scenarios that could be utilized with embodiments of the present invention, and embodiments of the present invention are contemplated for use with any appropriate trigger and sensor combinations.

At step 404, the system processes the initial speech from the user that is directed toward the pet. As detailed elsewhere herein, the processing of the speech can be performed on one or more computing devices, such as a local mobile device, a remote server or other computing device, on the device worn by the pet, or any combination thereof. In certain embodiments, selection of the appropriate device for processing can be performed by the initial device receiving the speech. For instance, a short command or statement may be easily processed on a local device, whereas a more complicated communication may require additional processing power and best for remote processing. Additionally, in certain embodiments, the speech may be converted to text on a local device and then transmitted to a remote computing device for processing, as text and other structured data can be less resource intensive than raw audio.

At step 406, the system is configured to perform a sentiment analysis on the speech. In this manner, the system is configured to determine the nature of the speech directed at the pet. Detectable sentiment can include, but is not limited to, prosody, tempo, intonation, stress, pauses, breath rate, voice quality, syntax, diction, phraseology, register, and any combination thereof. One of ordinary skill in the art would appreciate that there are many data points on which speech can be analyzed, such as pragmatics, discourse structure, sociolinguistics, emotional valence, cognitive load, fundamental frequency, energy, amplitude envelope, jitter, shimmer, Mel-frequency cepstral coefficients (MFCCs), voice activity detection, or any combination thereof.

Depending on the sensors available, the system may be configured to use other data for sentiment analysis as well, such as facial cues and body language (e.g., from visual/optical sensors), and heart rate and variability (e.g., from sensors on health devices and other wearables). One of ordinary skill in the art would appreciate that there are numerous sensors that could provide data to the system for use in processing sentiment analysis, and embodiments of the present invention are contemplated for use with any appropriate sensors.

At step 408, the system processes the state of the pet, based on available sensor data. In certain rudimentary embodiments, there may be no sensors, and the system will base generation on no or limited analysis of the status of the pet. In other embodiments, the system may be limited to audio data, whether from a wearable device on the pet, or from a computing device of the user (e.g., smartphone microphone). In further embodiments, the system may be configured to use one or more additional sensors for detecting a state of the pet. For instance, a wearable device on the pet may detect certain bioinformatic data, such as heart rate, respiratory rate, movement (e.g., via accelerometers), or any combination thereof. One of ordinary skill in the art would appreciate that there are numerous types of bioinformatic sensors that could be used with embodiments of the present invention, and embodiments of the present invention are contemplated for use with any such sensors.

At step 410, the system generates a response to the processed speech. In a preferred embodiment of the present invention, generation of a response may utilize data taken in and processed from the initial captured speech, the sentiment analysis of the speech, and the state of the animal. In certain embodiments, not just the choice of response as developed by the AI/ML components of the system, but the tone and other speech characteristics (e.g., prosody, tempo, intonation, stress, pauses, breath rate, voice quality, syntax, diction, phraseology, register) can be generated and tailored to be in line with other data being processed. For instance, if the system determines a pet is showing excitement characteristics (e.g., elevated heart and respiratory rates) through the data collected at the detection of the animal state step, the system may generate a response that matches the pet's characteristics, and the tonal qualities and other speech characteristics may match that state.

Once the response is generated, at step 412, the system is configured to engage in playback/deployment of audio associated with the response. In certain embodiments, the system may play the audio associated with the response at a predetermined audio level. In other embodiments, the system may utilize other input or sensor data to adjust the volume of the playback according to received input or sensor data. For instance, if the device has an optical sensor that can detect the pet owner is further away, or closer than a predetermined distance, the system may be configured to increase or decrease the volume in order to account for the distance. Other examples include altering the audio volume of the response based on the level of ambient noise in an environment, such as increasing the playback volume in loud environments, and reducing the volume of playback in quiet environments. One of ordinary skill in the art would appreciate that there are numerous input and sensors that could be used to determine the nature of a present environment, and make volume adjustments accordingly. After completion of playback of the response, the process terminates at step 414.

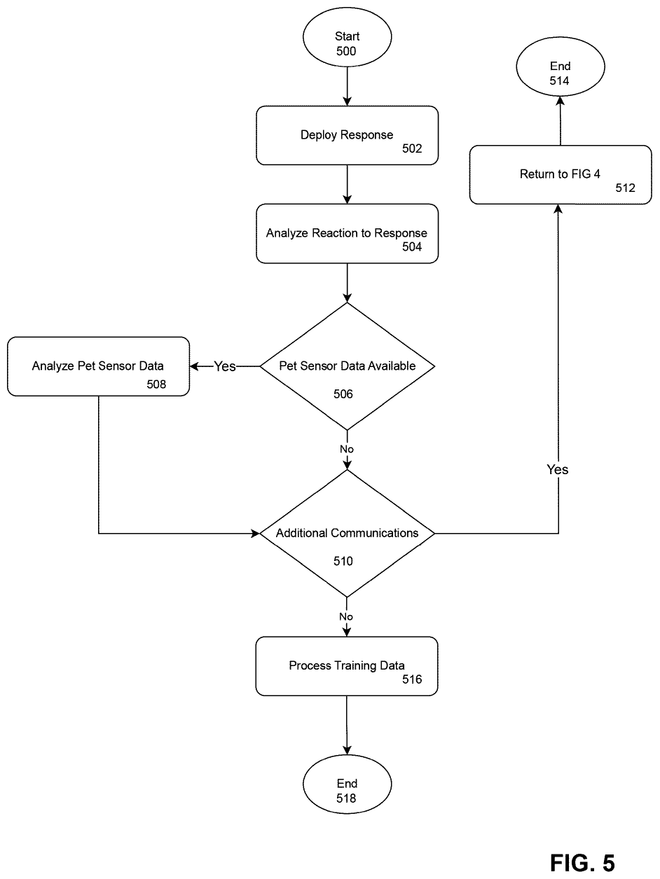

Turning now to FIG. 5, an exemplary method for emulating human conversations with animals, in accordance with an embodiment of the present invention, is shown. The process starts at step 500 with the system being engaged to analyze after effects of a deployed response, with the deployed response occurring at step 502. Once deployed, the system may be configured to receive data from any available inputs or sensors associated with the system, such as sensors or input devices on the speaker device, or from an application on a user's computing device. For example, the system may use a microphone on the speaker device to determine the user's response, such as by analyzing voice characteristics, including changes that would denote a happy, satisfied or unsatisfied response to the deployed response. In other examples, the system may utilize visual sensors, such as an optical sensor on the speaker device, or the camera components on a user's computing device, to detect and analyze changes in the user's affect, such as smiles, frowns, enlarged eyes/pupils, furrowing of brow, or any other physical change that could be observed by the optical sensor. One of ordinary skill in the art would appreciate that there are numerous sensors and inputs that could be utilized, such as data from wearable devices on the user, or additional sensors on the user or pet.

At step 506, the system is configured to determine if there is additional data to be retrieved related to the pet, such as sensors for detecting changes in affect of the pet, including, but not limited to, accelerometers, heart and respiratory rate sensors, microphones, optical sensors, electrochemical sensors, infrared sensors or any combination thereof. If so, the system collects and analyzes the additional data at step 508.

If no additional pet sensor data is available, or if the pet sensor data was collected and processed at step 508, the process then determines if additional communications are warranted/desired at step 510. If additional communications are desired, the process moves to step 512 and repeats the method detailed in FIG. 4. This process can loop as many times as the user desires to engage the pet and the system for communication purposes.

If no further communications are desired, at step 516, the system processes the outcome and results of each round of communications between the user and the pet in order to improve the models used with the system. In cases where responses elicited the desired outcome from the user and/or pet, the system may incorporate and learn from the data and retrain its model accordingly. Likewise, with less desirable outcomes, the system can leverage the data to retrain and update its models in order to attempt to optimize positive results occurring more frequently over time. This is advantageous, as each individual pet and user combo are going to have a unique bond and communication style, and the system will need to train and learn according to its users and related pets.

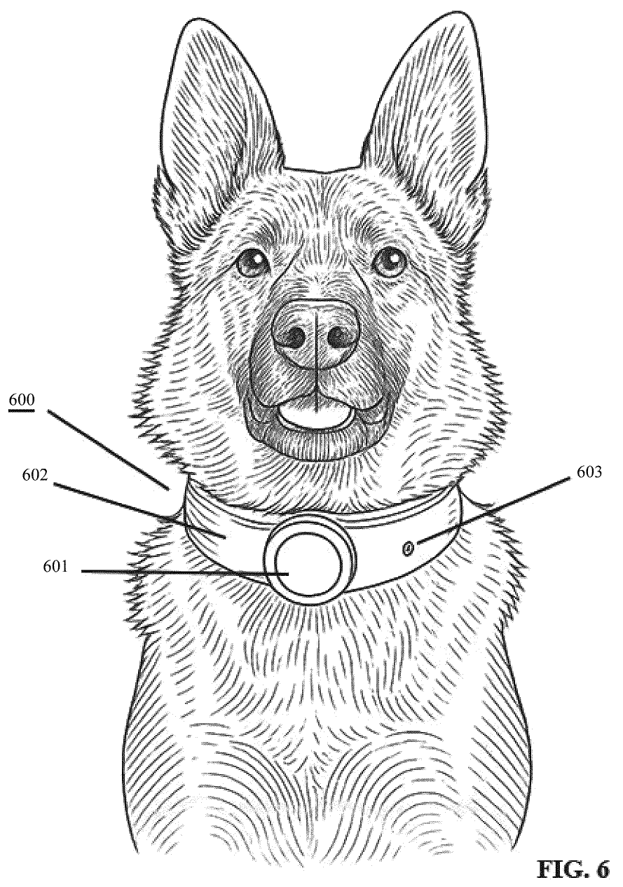

Turning now to FIG. 6 is an illustration of an exemplary device for emulating human conversations with animals as being worn by a canine, in accordance with an embodiment of the present invention. In this embodiment, a speaker device 600 is being worn by a canine pet. The speaker device 600 comprises a speaker and control unit 601, an attachment feature 602 comprising a detachable collar that secures the speaker device 600 to the pet, and a sensor component 603.

In this configuration, the speaker and control unit 601 is configured to preferably be located on a forward face of the pet, such that the sensors and speaker are in alignment with a direction the pet is facing. In other embodiments, the speaker and control unit 601 may be configured to be on a separate portion of the pet, such as flank, back, head or limb.

According to a preferred embodiment of the present invention, the attachment feature 602 may comprise a collar, vest or other wearable article that allows for the speaker device 600 to be retained on or worn by the pet. In preferred embodiments, the attachment feature 602 is configured to remain in a desired position on the pet (e.g., forward facing at all times), so that the data, input/output and sensor components in both the speaker and control unit 601 and the sensor component 603 are optimally aligned to receive and transmit data as needed.

According to an embodiment of the present invention, the sensor component 603 may be configured to provide additional input and/or output from the speaker device 600. In certain embodiments, the sensor component 603 may be distal from the speaker and control unit 601, in order to provide additional sensor data that may not be available or may be more optimally detected at a location distal from the speaker and control unit 601. For instance, a sensor component 603 could be located closer to an artery or other vascular area on a pet that would allow for detection of heart rate and/or respiratory rate. In other embodiments, the sensor component 603 may be located on a portion of a pet that may have less obstructions (e.g., an area on the pet with less fur, hair or feathers). In still further embodiments, there could be multiple sensor components 603 located at various locations on the speaker device 600, or otherwise on a separate wearable or detachable device (not shown in drawings). One of ordinary skill in the art would appreciate that there are numerous types of configurations that could be utilized with embodiments of the present invention, and embodiments of the present invention are contemplated for use with any appropriate configuration.

Further, in certain embodiments, the sensor component may be connected to the speaker and control unit 601 of the speaker device 600 in a variety of manners. For instance, the connection could be a wired connection, with wires running in the attachment feature 602. In other embodiments, the connection could be wireless, such as via Bluetooth components, Near Field Communication (NFC) components, WIFI components, cellular components, or any combination thereof. One of ordinary skill in the art would appreciate that there are numerous types of communications components that could be utilized with embodiments of the present invention, and embodiments of the present invention are contemplated for use with any appropriate communications component.

The communications means of the system may be any circuitry or other means for communicating data over one or more networks or to one or more peripheral devices attached to the system, or to a system module or component. Appropriate communications means may include, but are not limited to, wireless connections, wired connections, cellular connections, data port connections, Bluetooth® connections, near field communications (NFC) connections, or any combination thereof. One of ordinary skill in the art will appreciate that there are numerous communications means that may be utilized with embodiments of the present disclosure, and embodiments of the present disclosure are contemplated for use with any communications means.

Traditionally, a computer program includes a finite sequence of computational instructions or program instructions. It will be appreciated that a programmable apparatus or computing device can receive such a computer program and, by processing the computational instructions thereof, produce a technical effect.

A programmable apparatus or computing device includes one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. It will be understood that a computing device can include a computer-readable storage medium and that this medium may be internal or external, removable and replaceable, or fixed. It will also be understood that a computing device can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the disclosure as claimed herein could include an optical computer, quantum computer, analog computer, or the like.

Regardless of the type of computer program or computing device involved, a computer program can be loaded onto a computing device to produce a particular machine that can perform any and all of the depicted functions. This particular machine (or networked configuration thereof) provides a technique for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. Illustrative examples of the computer readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A data store may be comprised of one or more of a database, file storage system, relational data storage system or any other data system or structure configured to store data. The data store may be a relational database, working in conjunction with a relational database management system (RDBMS) for receiving, processing and storing data. A data store may comprise one or more databases for storing information related to the processing of moving information and estimate information as well one or more databases configured for storage and retrieval of moving information and estimate information.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software components or modules, or as components or modules that employ external routines, code, services, and so forth, or any combination of these. All such implementations are within the scope of the present disclosure. In view of the foregoing, it will be appreciated that elements of the block diagrams and flowchart illustrations support combinations of means for performing the specified functions, combinations of steps for performing the specified functions, program instruction technique for performing the specified functions, and so on.

It will be appreciated that computer program instructions may include computer executable code. A variety of languages for expressing computer program instructions are possible, including without limitation C, C++, Java, JavaScript, assembly language, Lisp, HTML, Perl, and so on. Such languages may include assembly languages, hardware description languages, database programming languages, functional programming languages, imperative programming languages, and so on. In some embodiments, computer program instructions can be stored, compiled, or interpreted to run on a computing device, a programmable data processing apparatus, a heterogeneous combination of processors or processor architectures, and so on. Without limitation, embodiments of the system as described herein can take the form of web-based computer software, which includes client/server software, software-as-a-service, peer-to-peer software, or the like.

In some embodiments, a computing device enables execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. The thread can spawn other threads, which can themselves have assigned priorities associated with them. In some embodiments, a computing device can process these threads based on priority or any other order based on instructions provided in the program code.

Unless explicitly stated or otherwise clear from the context, the verbs “process” and “execute” are used interchangeably to indicate execute, process, interpret, compile, assemble, link, load, any and all combinations of the foregoing, or the like. Therefore, embodiments that process computer program instructions, computer-executable code, or the like can suitably act upon the instructions or code in any and all of the ways just described.

In some embodiments, the system includes a caching mechanism to store responses for common pet interactions (e.g., “sit,” “good boy,” or “want a treat?”), reducing the need to repeatedly use potentially costly processing steps, including, but not limited to, for example, LLM token usage or speech-to-text and text-to-speech conversions. By checking the cache first, the system includes the potentially for skipping these potentially costly processing steps for frequent responses, directly deploying a pre-stored audio response, potentially minimizing computational overhead and associated expenses. This response caching module could be a database or memory cache storing mappings of common speech inputs, pet-specific data, and pre-generated audio responses. One of ordinary skill in the art would appreciate that there are numerous types of caching configurations that could be utilized with embodiments of the present invention, and embodiments of the present invention are contemplated for use with any appropriate configuration.

The functions and operations presented herein are not inherently related to any particular computing device or other apparatus. Various general-purpose systems may also be used with programs in accordance with the teachings herein, or it may prove convenient to construct more specialized apparatus to perform the required method steps. The required structure for a variety of these systems will be apparent to those of ordinary skill in the art, along with equivalent variations. In addition, embodiments of the disclosure are not described with reference to any particular programming language. It is appreciated that a variety of programming languages may be used to implement the present teachings as described herein, and any references to specific languages are provided for disclosure of enablement and best mode of embodiments of the disclosure. Embodiments of the disclosure are well suited to a wide variety of computer network systems over numerous topologies. Within this field, the configuration and management of large networks include storage devices and computing devices that are communicatively coupled to dissimilar computing and storage devices over a network, such as the Internet, also referred to as “web” or “world wide web”.

Throughout this disclosure and elsewhere, block diagrams and flowchart illustrations depict methods, apparatuses (e.g., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general purpose hardware and computer instructions; and so on-any and all of which may be generally referred to herein as a “component”, “module,” or “system.”

While the foregoing drawings and description set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more sub-steps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

The functions, systems and methods herein described could be utilized and presented in a multitude of languages. Individual systems may be presented in one or more languages and the language may be changed with ease at any point in the process or methods described above. One of ordinary skill in the art would appreciate that there are numerous languages the system could be provided in, and embodiments of the present disclosure are contemplated for use with any language.

While multiple embodiments are disclosed, still other embodiments of the present disclosure will become apparent to those skilled in the art from this detailed description. There may be aspects of this disclosure that may be practiced without the implementation of some features as they are described. It should be understood that some details have not been described in detail in order to not unnecessarily obscure the focus of the disclosure. The disclosure is capable of myriad modifications in various obvious aspects, all without departing from the spirit and scope of the present disclosure. Accordingly, the drawings and descriptions are to be regarded as illustrative rather than restrictive in nature.

What is claimed is:

1. A system for creating interactive, real-time conversations between pets and their owners via a computerized system, the system comprising:
a pet communication device, comprising a processor, a data communications module and a pet communication module stored in non-transitory memory and configured to instruct said processor to:
detect engagement of a directed conversation;
process initial speech associated with the directed conversation;
determine a sentiment analysis of the initial speech;
generate a response to the initial speech, based at least in part on the sentiment analysis and known data points associated with a pet or owner associated with the pet communication device; and
deploy an audio response, based on the response to the initial speech, to an audio playback component.

2. The system of claim 1, wherein the pet communication device further comprises a sensor module, and wherein the pet communication module is further configured to instruct said processor to:
receive sensor data associated with the pet from the sensor module;
process an animal state, based at least in part on the sensor data; and
incorporate said animal state into the known data points associated with the pet.

3. The system of claim 2, wherein the sensor module is selected from the group comprising, an optical sensor module, an audio sensor module, an infrared sensor module, accelerometer module, a heart rate sensor module, a respiratory rate sensor module, an electrochemical sensor module, or a thermal sensor module.

4. The system of claim 2, wherein data related to the animal state is based at least in part on a delta between a first animal state occurring prior to receipt of the initial speech and a second animal state occurring after the receipt of the initial speech.

5. The system of claim 2, wherein the pet communication module is further configured to instruct said processor to:
detect changes in affect of a user, based at least in part on said audio response;
analyze the changes in affect for desirability of outcome; and
update a data model for use in generating response to user to pet speech based at least in part on the desirability of outcome.

6. The system of claim 1, wherein the pet communication module is further configured to instruct said processor to:
transmit, via the data communications module, the initial speech to a remote computing device for processing; and
receive, via the data communications module, the response to the initial speech, which was processed on the remote computing device.

7. The system of claim 1, wherein the audio playback component is located on a user device.

8. The system of claim 1, wherein the audio playback component is located on the pet communication device.

9. The system of claim 1, wherein the pet communication module stores a conversation history to generate context-aware responses.

10. The system of claim 1, further comprising a response caching module configured to store pre-generated responses to frequently occurring pet interactions, wherein the pet communication module is further configured to instruct said processor to:
determine whether the processed initial speech corresponds to a common interaction based on a predefined set of frequent speech patterns or commands;
retrieve a cached response from the response caching module that matches the common interaction and the known data points associated with the pet;
deploy the cached audio response to the audio playback component without engaging some computational processing, including but not limited to, for example, engaging a large language model (LLM) for response generation or any other computational processes, such as any speech-to-text conversion, or text-to-speech conversion processes.

11. The system of claim 1, further comprising a neural interface module configured to:
receive neural signals from a neurotechnology implant associated with the pet;
process the neural signals to determine the pet's mental state or intended response;
incorporate the processed neural data into the known data points associated with the pet for generating the response to the initial speech.

12. The system of claim 11, wherein the neural signals are processed using machine learning algorithms trained on datasets of pet neural activity correlated with observable behaviors or physiological states.

13. A computerized method creating interactive, real-time conversations between pets and their owners, the method comprising the steps of:
detecting, via a pet communication device, engagement of a directed conversation;
processing, via a pet communication device, initial speech associated with the directed conversation;
determining, via a pet communication device, a sentiment analysis of the initial speech;
generating, via a pet communication device, a response to the initial speech, based at least in part on the sentiment analysis and known data points associated with a pet or owner associated with the pet communication device; and
deploying an audio response, based on the response to the initial speech, to an audio playback component.

14. The method of claim 13, further comprising:
receiving sensor data associated with the pet from the sensor module;
processing an animal state, based at least in part on the sensor data; and
incorporating said animal state into the known data points associated with the pet.

15. The method of claim 14, wherein the sensor module is selected from the group comprising, an optical sensor module, an audio sensor module, an infrared sensor module, accelerometer module, a heart rate sensor module, a respiratory rate sensor module, an electrochemical sensor module, or a thermal sensor module.

16. The method of claim 13, wherein data related to the animal state is based at least in part on a delta between a first animal state occurring prior to receipt of the initial speech and a second animal state occurring after the receipt of the initial speech.

17. The method of claim 14, further comprising the steps of:
detecting changes in affect of a user, based at least in part on said audio response;
analyzing the changes in affect for desirability of outcome; and
updating a data model for use in generating response to user to pet speech based at least in part on the desirability of outcome.

18. The method of claim 13, further comprising the steps of:
transmitting, via a data communications module, the initial speech to a remote computing device for processing; and
receiving, via the data communications module, the response to the initial speech, which was processed on the remote computing device.

19. The method of claim 13, wherein the audio playback component is located on a user device.

20. The method of claim 13, wherein the audio playback component is located on the pet communication device.

21. The method of claim 13, wherein the pet communication module stores a conversation history to generate context-aware responses.

22. The method of claim 13, further comprising a response caching module configured to store pre-generated responses to frequently occurring pet interactions, wherein the pet communication module is further configured to instruct said processor to:
determine whether the processed initial speech corresponds to a common interaction based on a predefined set of frequent speech patterns or commands;
deploy the cached audio response to the audio playback component without engaging some computational processing, including but not limited to, for example, engaging a large language model (LLM) for response generation or any other computational processes, such as any speech-to-text conversion, or text-to-speech conversion processes.

23. The method of claim 13, further comprising a neural interface module configured to:
receive neural signals from a neurotechnology implant associated with the pet;
process the neural signals to determine the pet's mental state or intended response;
incorporate the processed neural data into the known data points associated with the pet for generating the response to the initial speech.

24. The method of claim 13, wherein the neural signals are processed using machine learning algorithms trained on datasets of pet neural activity correlated with observable behaviors or physiological states.
